(19) United States
(12) Patent Application Publication
(10) Pub. No.: US 2024/0407966 A1
(43) Pub. Date: Dec. 12, 2024

(54) TISSUE REJUVENATION BY RESTRICTED BLOOD FLOW AND THERMOTHERAPY

(71) Applicant: Moshe Ein-Gal, Ramat Hasharon (IL)

(72) Inventor: Moshe Ein-Gal, Ramat Hasharon (IL)

(21) Appl. No.: 18/332,898

(22) Filed: Jun. 12, 2023

(51) Int. Cl. A61H 7/00; A61H 19/00

(52) U.S. Cl. CPC A61H 7/001; A61H 19/32; A61H 2201/0207; A61H 2205/022; A61H 2209/00

(57) ABSTRACT

A method for tissue rejuvenation includes using a combination of hypoxia-causing blood-flow restriction and thermotherapy applied to a treated body portion to cause tissue rejuvenation in the treated body portion, the hypoxia-causing blood-flow restriction being done by impeding blood circulation to the treated body portion and the thermotherapy being done by applying heat to the treated body portion.

### FIELD OF THE INVENTION

The present invention relates generally to tissue rejuvenation by a combination of blood flow restriction and thermotherapy.

### BACKGROUND OF THE INVENTION

Tissue rejuvenation (the terms “rejuvenation” and “regeneration” are used interchangeably throughout) involves restoring or regenerating damaged or aging tissues, such as by stimulating the body's natural healing mechanisms to repair and renew cells, leading to improved tissue structure and function. Promoting tissue rejuvenation includes medical interventions, lifestyle changes, and regenerative medicine techniques.

Regenerative medicine focuses on using stem cells, growth factors, and other biological materials to stimulate tissue regeneration.

Platelet-rich plasma (PRP) therapy involves injecting a concentrated solution of the patient's own platelets (containing growth factors) into the injured or aging tissue.

Heating by optical, electromagnetic or ultrasound energy can also be used to rejuvenate the skin by promoting collagen production and stimulating the growth of new, healthier skin cells. Skin rejuvenation can be used to reduce wrinkles, improve skin texture, and treat scars or blemishes.

A healthy diet and appropriate supplementation can support tissue rejuvenation by providing essential nutrients for cell growth and repair. For example, collagen supplements are often used to promote skin elasticity and joint health.

Regular exercise and physical therapy can help stimulate blood flow, increase oxygenation, and promote tissue healing. These activities can improve muscle strength, flexibility, and overall tissue health.

Adopting a healthy lifestyle, including proper sleep, stress management, and avoiding harmful habits like smoking or excessive alcohol consumption as well as other lifestyle changes, can contribute to tissue rejuvenation. Chronic stress and unhealthy habits can negatively affect tissue health and impede the body's healing processes.

Key elements of tissue rejuvenation are angiogenesis and collagen production. Angiogenesis, in which new blood vessels are formed from existing ones, plays a crucial role in various physiological and pathological processes, including wound healing and tissue rejuvenation. Heat has been shown to induce the release of various growth factors and cytokines that are involved in angiogenesis. For example, heat stress can stimulate the production of vascular endothelial growth factor (VEGF), which is a potent inducer of angiogenesis; it acts on nearby blood vessels, promoting their growth and sprouting. Additionally, hyperthermia-induced vasodilation can lead to the recruitment of endothelial progenitor cells, which are involved in blood vessel formation.

Heat stimulates production of collagen, which is the primary structural protein in skin, tendons, ligaments, and other connective tissues. Hyperthermia activates fibroblasts, which are the cells responsible for producing collagen. Heat stress triggers the release of certain growth factors which can activate fibroblasts and promote collagen production. Hyperthermia induces production of heat shock proteins (which play a crucial role in collagen synthesis) and can enhance collagen cross-linking, which is the formation of chemical bonds between collagen molecules.

Restricted blood flow causes hypoxia (defined as a deficiency in oxygen supply) known to promote angiogenesis by triggering physiological responses aimed at restoring oxygen delivery. One of these responses is the release of certain signaling molecules, such as VEGF, by cells in the hypoxic region.

The vascular network delivers oxygen and nutrients to all cells within the body. Most transcriptional responses to low oxygen are mediated by hypoxia-inducible factors (HIFs), highly conserved transcription factors that control the expression of numerous angiogenic, metabolic, and cell cycle genes. Accordingly, the HIF pathway is currently viewed as a master regulator of angiogenesis. HIF modulation may provide therapeutic benefit for a wide array of pathologies, including cancer, ischemic heart disease, peripheral artery disease, wound healing, and neovascular eye diseases. Hypoxia promotes vessel growth by upregulating multiple pro-angiogenic pathways that mediate key aspects of endothelial, stromal, and vascular support cell biology.

If some tissues do not receive enough oxygen (hypoxia), cells in the affected area send out chemical signals that cause angiogenesis to begin.

In summary, hypoxia promotes angiogenesis through the release of signaling molecules like VEGF, which stimulate the growth of new blood vessels from existing ones. Acute hypoxia lasts a few minutes to a few hours.

### SUMMARY OF THE INVENTION

The present invention seeks to provide a method and device for tissue rejuvenation by a combination of thermotherapy and hypoxia-causing blood-flow restriction (referred to below as “flow restriction” for short). The prior art has not suggested combining flow restriction and thermotherapy. Furthermore, it has been surprisingly found that the combination of flow restriction and thermotherapy provides synergistic results; that is, the effectiveness of the combination of flow restriction and thermotherapy is significantly better than the combined effectiveness of just flow restriction alone and just thermotherapy alone.

There is provided in accordance with a non-limiting embodiment of the present invention a method for tissue rejuvenation including using a combination of flow restriction and thermotherapy applied to a treated body portion to cause tissue rejuvenation in the treated body portion, the flow restriction being done by impeding blood circulation to the treated body portion and the thermotherapy being done by applying heat to the treated body portion.

In accordance with a non-limiting embodiment of the present invention the treated body portion is a hand, an arm, a foot, a leg, a neck, a face, a scalp, a vagina, a skin portion, a penis, a penis crus, or pelvic muscles.

In accordance with a non-limiting embodiment of the present invention application of the flow restriction and the thermotherapy to the treated body portion is consecutively implemented in several cycles, and the blood circulation to the treated body portion is restored between consecutive cycles while the thermotherapy continues.

In accordance with a non-limiting embodiment of the present invention the thermotherapy is applied using more than one type of energy, including electromagnetic energy, optical energy, radiofrequency energy, acoustic energy, or fluid conduction thermal energy.

In accordance with a non-limiting embodiment of the present invention the thermotherapy is aided by another type of energy, including electromagnetic energy, optical energy, radiofrequency energy, acoustic energy, or fluid conduction thermal energy.

In accordance with a non-limiting embodiment of the present invention the treated body portion is a penis, and the method further includes stretching the penis or causing the penis to be erect, blocking the blood circulation to the penis, and applying thermotherapy to the penis, and the method is used to treat erectile dysfunction.

In accordance with a non-limiting embodiment of the present invention the treated body portion is a face, and the method includes blocking the blood circulation to the face by applying external pressure using a pressure-applying facial mask, and the pressure is applied by pulling and/or pushing the facial mask toward the face using. for example, a spring, an elastic band, gravitation, a pneumatic assembly, or an electric motor.

In accordance with a non-limiting embodiment of the present invention the treated body portion is a skin portion, and the method further includes blocking the blood circulation to the skin portion by a pressure-applying skin mask and forcing treatment lotion into the skin portion by the external pressure exerted by the skin mask on the skin portion.

There is provided in accordance with a non-limiting embodiment of the present invention a device for tissue rejuvenation including a blood flow constrictor, a heat source, and a controller coupled to the blood flow constrictor and to the heat source, for applying a combination of flow restriction and thermotherapy to a treated body portion to cause tissue rejuvenation in the treated body portion.

There is provided in accordance with a non-limiting embodiment of the present invention a device for tissue rejuvenation including a pressure-applying body portion mask for flow restriction, a heat source, and a controller coupled to the body portion mask and to the heat source, for applying a combination of flow restriction and thermotherapy to the treated body portion to cause tissue rejuvenation in the treated body portion.

In accordance with a non-limiting embodiment of the present invention a temperature sensor and a pressure sensor are provided which are in operative communication with a controller, and the controller is configured to control operative parameters of the blood flow constrictor or the body portion mask and the heat source as a function of feedback from the temperature sensor and the pressure sensor.

In accordance with a non-limiting embodiment of the present invention the blood flow constrictor includes a constriction ring or a pressure-applying body portion mask.

### BRIEF DESCRIPTION OF THE DRAWINGS

The present invention will be understood and appreciated more fully from the following detailed description taken in conjunction with the drawings in which:

FIG. 1 is a simplified illustration of a device for tissue rejuvenation by a combination of flow restriction and thermotherapy, in accordance with a non-limiting embodiment of the present invention, whereas the blood flow constrictor is a constricting ring or a pressure-applying body portion mask.

FIG. 2 is a simplified block diagram of a method for tissue rejuvenation by a combination of flow restriction and thermotherapy, in accordance with a non-limiting embodiment of the present invention.

### DETAILED DESCRIPTION OF EMBODIMENTS

As mentioned above, the invention encompasses thermotherapy assisted by flow restriction and heat-assisted hypoxia.

Without limiting the invention to any theory, it is possible that the elimination or significant reduction of blood circulation in the organ, appendage or body portion being treated, causes the temperature distribution of the treated body portion to be solely determined by tissue parameters and deposited energy; the thermal energy of the blood flow does not negatively affect the temperature distribution in the treated body portion. Consequently, the steady-state temperature distribution in the treated body portion is better achieved both spatially (more uniform) and temporally (faster).

Again, without limiting the invention to any theory, it is possible that heating assists tissue rejuvenating hypoxia, because the body portion senses or detects the increased temperature, which increases the demand for oxygen uptake by the heated tissue. The increased demand for oxygen may accelerate triggering the angiogenic processes.

Due to the mutual assistance, the combination of flow restriction and thermotherapy provides synergistic results; that is, the effectiveness of the combination of flow restriction and thermotherapy is significantly better than the combined effectiveness of just flow restriction alone and just thermotherapy alone.

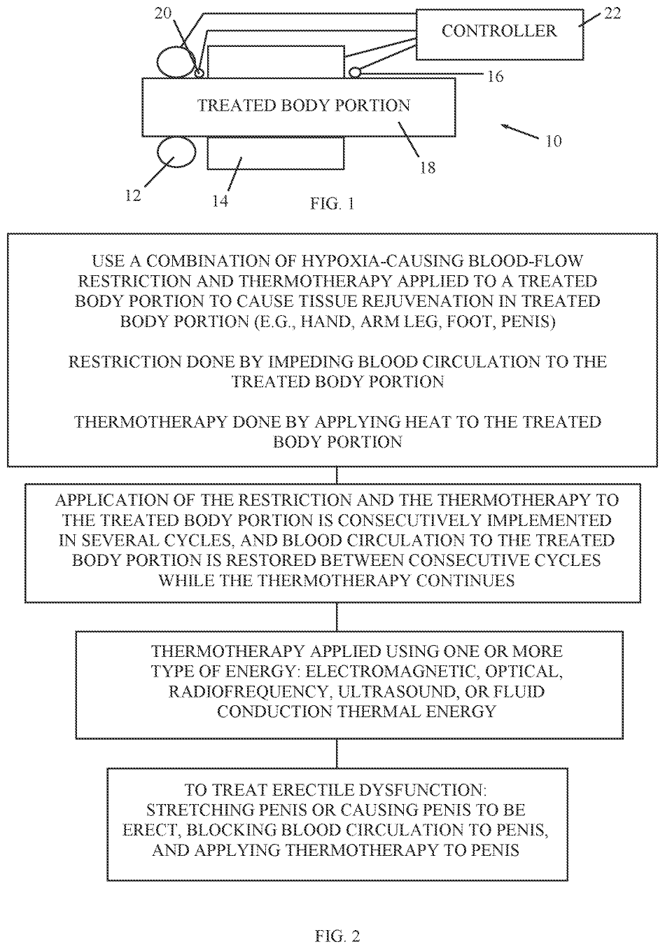

Reference is now made to FIG. 1, which illustrates a device 10 for tissue rejuvenation by a combination of hypoxia-causing blood-flow restriction and thermotherapy, in accordance with a non-limiting embodiment of the present invention.

Device 10 may include a blood flow constrictor 12, such as but not limited to, a constriction ring used for treating erectile dysfunction, an elastic band, tourniquet and others. Device 10 may include a heat source 14, such as but not limited to, electrical resistance heater, thermoelectric heater, chemical heater, optical heater (e.g., laser, LED, or flash lamp), infrared, acoustic, electromagnetic, radiofrequency, fluid conductor of thermal energy, and others.

In accordance with another non-limiting embodiment of the invention, blood flow constrictor 12 may be a pressure-applying body portion mask. For example, the pressure-applying body portion mask may be a pressure-applying facial mask, and the pressure is applied by pulling and/or pushing the facial mask toward the face using. for example, a spring, an elastic band, gravitation, a pneumatic assembly, or an electric motor. In such an application of the invention, the treated body portion is a face, and the method includes blocking the blood circulation to the face by applying external pressure using the pressure-applying facial mask.

In accordance with another non-limiting embodiment of the present invention, the pressure-applying body portion mask (blood flow constrictor 12) may be a pressure-applying skin mask. In such an application of the invention, the treated body portion is a skin portion, and the method includes blocking the blood circulation to the skin portion by using the pressure-applying skin mask, and forcing treatment lotion into the skin portion by the external pressure exerted by the skin mask on the skin portion.

Device 10 may include a temperature sensor 16, such as but not limited to, a thermistor or thermocouple and others, which senses the temperature of the treated body portion 18 which is being treated. Device 10 may include a pressure sensor 20, such as but not limited to, a strain gauge, piezoelectric pressure sensor, piezoresistive strain gauge, capacitance pressure sensor and others, which senses the pressure being applied by the blood flow constrictor 12 to the treated body portion 18 The temperature sensor 16 and the pressure sensor 20 may communicate with a controller 22, which controls the operative parameters of blood flow constrictor 12 and heat source 14 as a function of feedback from temperature sensor 16 and pressure sensor 20. Controller 22 may include a display and user interface, as is well known. The pressure-applying body portion mask mentioned above may also cooperate with the thermotherapy source, temperature sensor, pressure sensor, and controller.

Reference is now made to FIG. 2, which illustrates a method for tissue rejuvenation by a combination of flow restriction and thermotherapy, by using the device of FIG. 1, in accordance with a non-limiting embodiment of the present invention.

It is contemplated that the tissue rejuvenating treatment may be implemented in several sessions during a treatment time of at least one week. In a session, the treatment may include short cycles separated by breaks of restored blood circulation and continuing thermotherapy. Short cycles and associated breaks allow safe repeated hypoxia applications. The benefits of the cycles include dilation of blood-circulated tissue during the breaks, and a safe long treatment time in a session by increasing the number of cycles.

Thermotherapy may be achieved by using delivery of various energies, such as but not limited to, electromagnetic or optical (for non-contact delivery), and radiofrequency, acoustic, or fluid conduction thermal energy (for contact delivery). The relationship between the delivered energy and the organ temperature distribution is governed by the temporal-spatial bio-heat equation, where blood circulation is an important contributor to energy flowing to/from the organ. Blocked blood circulation eliminates, or significantly reduces, the circulation component in the bio-heat equation, thus enabling efficient reaching of desired steady-state temperature distribution in the organ. Temperature ranges should be selected to prevent charring or other tissue damage, depending on which tissue is being treated.

Organ hypoxia may be achieved by reducing blood circulation to the organ. Appendage hypoxia can be achieved by applying external pressure on vessels circulating blood to the appendage. An example of blocking blood circulation to an appendage is by placing a constricting ring on the base of an erected penis for trapping the blood in the penis so as to maintain the erection; the blood circulation has to be restored for safety reasons in no more than 30 minutes.

Erected or stretched penis is preferred when applying the method for ED treatment. Erection may be assisted by a vasodilating drug, such as but not limited to, phosphodiesterase type 5 (PDE5) inhibitors, injection to the cavernous bodies or by applying vacuum for filling the penis with blood and placing a constricting ring for blood trapping in the penis. Maintaining a stretched penis can be mechanically implemented, for example, by pulling the penis with a stretchable string or by rigidly scaffolding the stretched penis. Constriction and release of the penis base may be implemented by a tubular inflatable ring, allowing the constriction and release to be executed by controlling the pressure supplied by a communicating pump to the tubular ring. This function is particularly useful for implementing cycles of blood flow blockage and restoration during a session.

The device of the invention may be a device for home-use, clinic, or hospital use according to cost, safety and usage simplicity.

What is claimed is:

1. A method for tissue rejuvenation comprising:
using a combination of hypoxia-causing blood-flow restriction and thermotherapy applied to a treated body portion to cause tissue rejuvenation in said treated body portion, the hypoxia-causing blood-flow restriction being done by impeding blood circulation in said treated body portion and the thermotherapy being done by applying heat to said treated body portion.

2. The method according to claim 1, wherein said treated body portion is a hand, an arm, a foot, a leg, a neck, a face, a scalp, a vagina, a skin portion, a penis, a penis crus, or pelvic muscles.

3. The method according to claim 1, wherein application of the hypoxia-causing blood-flow restriction and the thermotherapy to said treated body portion is consecutively implemented in several cycles, and the blood circulation to said treated body portion is restored between consecutive cycles while the thermotherapy continues.

4. The method according to claim 1, wherein the thermotherapy is applied using more than one type of energy, including at least two of electromagnetic energy, optical energy, radiofrequency energy, acoustic energy, or fluid conduction thermal energy.

5. The method according to claim 1, wherein additional type of energy is delivered to the body portion, including electromagnetic energy, optical energy, radiofrequency energy, acoustic energy, or fluid conduction thermal energy.

6. The method according to claim 1, wherein said treated body portion is a penis, and the method further comprises stretching the penis or causing the penis to be erect, blocking the blood circulation to the penis, and applying thermotherapy to the penis, and the method is used to treat erectile dysfunction.

7. The method according to claim 1, wherein impeding blood circulation to said treated body portion comprises using a pressure-applying body portion mask which applies pressure to the treated body portion.

8. The method according to claim 1, wherein said pressure-applying body portion mask comprises a pressure-applying skin mask or a pressure-applying facial mask.

9. The method according to claim 8, wherein said pressure-applying body portion mask comprises a pressure-applying skin mask, and the method further includes blocking blood circulation to a skin portion by applying said pressure-applying skin mask to the skin portion, and forcing treatment lotion into the skin portion by external pressure exerted by the pressure-applying skin mask on the skin portion.

10. A device for tissue rejuvenation comprising:
a blood flow constrictor;
a heat source; and
a controller coupled to said blood flow constrictor and to said heat source, for applying a combination of hypoxia-causing blood-flow restriction and thermotherapy to a treated body portion to cause tissue rejuvenation in said treated body portion.

11. The device according to claim 10, wherein said heat source comprises at least one of an electromagnetic energy, an optical energy, a radiofrequency energy, an acoustic, or a fluid conduction thermal energy heat source.

12. The device according to claim 10, further comprising a temperature sensor and a pressure sensor which are in operative communication with said controller, and said controller is configured to control operative parameters of said blood flow constrictor and said heat source as a function of feedback from said temperature sensor and said pressure sensor.

13. The device according to claim 10, wherein said blood flow constrictor comprises a constriction ring or a pressure-applying body-portion mask.

14. The device according to claim 13, wherein said pressure-applying body portion mask comprises a pressure-applying skin mask or a pressure-applying facial mask.
